SEQUENCE LISTING (3 sequences)

SEQ ID NO: 1 - DNA, 15 bp, ARTIFICIAL SEQUENCE
  misc_binding (1)...(15): targeted DNA sequence
cacgttgaggggcat

SEQ ID NO: 2 - DNA, 15 bp, ARTIFICIAL SEQUENCE
  misc_binding (1)...(15): targeted DNA sequence
gcccgagaacatcat

SEQ ID NO: 3 - DNA, 15 bp, ARTIFICIAL SEQUENCE
  misc_binding (1)...(15): targeted DNA sequence
cctcgcagtttccat

(19) United States
(12) Patent Application Publication
(10) Pub. No.: US 2003/0125283 A1
(43) Pub. Date: Jul. 3, 2003

(54) Therapy of proliferative disorders by direct irradiation of cell nuclei with tritiated nuclear targetting agents

(76) Inventor: Robert A. Gatenby, Tucson, AZ (US)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/221,969

(22) PCT Filed: Mar. 16, 2001
(86) PCT No.: PCT/US01/08446

(51) Int. Cl. A61K 51/00; A61K 48/00

(52) U.S. Cl. 514/44; 424/1.73

(57) ABSTRACT

The present invention provides methods of treating proliferative disorders in vivo by the direct administration of tritium to target cell nuclei. Tritium is administered to target cell nuclei by a tritiated nuclear targeting agent, which is directed to the target cell nucleus where it associates with the cell's DNA. The close association of the tritiated nuclear targeting agent with the target cell DNA allows the low-energy beta particle emitted by the tritium to damage to the target cell DNA and kill the cell. Tritiated nuclear targeting agents can also be delivered to the target cells by structures such as liposomes, micelles and microcapsules.

The benefit of the filing dates of U.S. Provisional Application Serial No. 60/192,153, filed Mar. 24, 2000, and U.S. Provisional Application Serial No. 60/192,671, filed Mar. 28, 2000 is hereby claimed.

### FIELD OF THE INVENTION

This invention relates to the field of proliferative disease therapy with low-energy radionuclides, in particular the direct irradiation of tumor cell nuclei with tritiated nucleic acid precursors.

### BACKGROUND OF THE INVENTION

Proliferative disorders are characterized by the uncontrolled growth of cells of certain tissue type or types, and can be classified as cancerous or non-cancerous. Cancerous proliferative disorders are characterized by the uncontrolled growth of cells with a malignant phenotype, meaning that the cells evade both the normal controls on cell growth and position. Thus, the cells not only form cancerous lesions (e.g., tumors or neoplasms) but can invade underlying tissue or migrate to other areas of the body and establish cancerous lesions there. The process of malignant cell migration in cancerous proliferative disorders is called metastasis. Non-cancerous proliferative disorders are characterized by the uncontrolled growth of cells with a benign phenotype, meaning that the cells evade only the normal controls on growth, but cannot metastasize.

Vascular restenosis is a non-cancerous proliferative disease common in patients being treated for coronary artery disease (CAD). Typically, CAD is treated by coronary angioplasty, which is a non-surgical procedure designed to restore the patency of blocked or partially blocked coronary arteries. Typically, this procedure is carried out with a balloon catheter. Coronary angioplasty results in successful revascularization in more than 90% of coronary artery disease patients. More than 300,000 coronary angioplasty procedures were performed in the United States in 1990. However, the major limitation of coronary angioplasty is a 30-40% restenosis rate which occurs in the first six months following the procedure.

Vascular smooth muscle cell (VSMC) proliferation has been identified as playing an important role in the development of atherosclerosis and restenosis following coronary angioplasty. The presence of VSMCs has been confirmed in both types of lesions, and is due primarily to a change from a contractile to a synthetic phenotype in VSMCs. This phenotypic change is associated with VSMC proliferation, migration from media to intima, and the synthesis of extracellular matrix, all of which results in neointimal formation (narrowing of the artery). In contrast to atherosclerosis, where this process is extended over several decades, vascular restenosis represents an acute response to balloon injury culminating in a significant renarrowing by neointimal formation of an initially patent vessel in the course of a few months. Recent studies have also observed the same response to implanted brachytherapeutic stents. For example, Albiero et al. reported high (43% to 50%) restenosis rates and high need for target vessel revascularization at four to six months in patients with radioactive stents. Although the radioactive stents reduced in-stent hyperplasia in a dose-related manner and no in-stent restenosis was observed at the high radiation doses, restenosis was found at the proximal and distal edges of the stent. These restenotic lesions can be seen as a narrowing of the vessel at each end of the stent, producing what he termed a “candy-wrapper” appearance on an angiogram (Albiero et al., Procedural results and 30-day clinical outcome after implantation of beta-particle emitting radioactive stents in human coronary arteries. Abstract #2563 Presented at the XXth Congress of the Eur. Soc. Cardiol., Aug. 22-26, 1999 Vienna, Austria). It has been suggested that the restenoses are due to a combination of barotrauma from the balloon inflation used to implant the stent and the lower radiation dose present at the stent ends (Serruys P W et al. Beta-particle emitting radioactive stent to prevent restenosis. Abstract #2564 Presented at the XXth Congress of the Eur. Soc. Cardiol., Aug. 22-26, Vienna, Austria). Hence, it has become apparent that the elimination of proliferating VSMCs without resorting to brachytherapy techniques or further mechanical trauma to the vascular wall is necessary to control the restenosis process.

Proliferative disorders may be treated with ionizing radiation. Ionizing radiation is cytotoxic because it disrupts DNA either by direct impact on a component of the molecule or by generating free-radical intermediates which cause chemical damage to the DNA (Hall E J, Radiobiology for the Radiologist (4^{th} ed.), J.B. Lippincott Co., Philadelphia 1994, pp. 39-40). When sufficient radiation-induced DNA damage accumulates in a cell, the cell dies (Carrano A V (1973) “Chromosome Aberrations and Radiation-Induced Cell Death,” Mutat. Res 17: 355-366). The effect of ionizing radiation on other cell components is negligible in terms of inducing cell death. Thus, the cytoreductive effects of radiation therapy for proliferative disorders is dependent on how much of the radiation reaches the proliferating cell's nucleus.

The volume of a tissue consists primarily of extracellular space and cytoplasm. Therefore, radiation applied externally as a therapy for proliferative disorders is largely ineffective because the incident particles deposit their energy in structures outside the nucleus of the proliferative cells. To ensure that a sufficient amount of radiation reaches the proliferative cell nuclei, clinicians must use high doses of externally applied radiation. These high radiation doses can cause damage to surrounding normal tissue.

Applying the radiation source directly to the proliferative tissue, for example a tumor or restenotic plaque, can reduce the radiation dose absorbed by the surrounding normal tissue. The direct application of a therapeutic radiation source is called “brachytherapy.” Brachytherapy maximizes the dose absorbed by the proliferative tissue and reduces the radiation damage of the surrounding normal tissue.

Examples of brachytherapy techniques include the implantation of radionuclide-containing sources, e g., ¹²⁵I or ¹⁰³Pd “seeds” for treatment of prostate cancer. Brachytherapy is also used in the treatment of restenosis after the removal of vascular occlusions. Current treatment of restenoses include the temporary or permanent placement of a device containing a radioisotope source at the site of restenosis. For example, U.S. Pat. No. 5,059,166 to Fischell et al. discloses a stent where the radioisotope source is contained either in the surface coating of the stent or in the metal alloy that forms the stent. U.S. Pat. No. 5,199,929 to Dake et al. discloses a catheter with a radioisotope source permanently attached to the distal end. U.S. Pat. No. 5,899,882 to Waksman discloses a closed-end lumen catheter that contains strontium-90.

However, brachytherapy techniques still allow the majority of the radiation dose to be absorbed by non-nuclear structures in the proliferative cells. Thus, there is needed a proliferative disorder therapy system which delivers substantially all the applied radiation dose directly to the nuclei of proliferative cells.

Leclerc et al., in U.S. Pat. No. 5,821,354, discloses the delivery of high-energy radionuclides to tumor cell nuclei with short stretches of radiolabeled complementary DNA. However, particles emitted by the high-energy radionuclides generally have a penetration distance in tissue of several millimeters to several centimeters. A cell typically has a cytoplasmic diameter of about 20-40 microns, and nuclear diameter of 1-2 microns. Thus, most of the emitted particles in the method described by Leclerc et al. will escape the cell nucleus to expend their energy in the cytoplasm or extracellular space. High energy radionuclides delivered to cell nuclei, for example in tissue characteristic of proliferative disorders, will therefore expend their energy largely outside the nucleus.

Therefore, what is needed is a therapy system for proliferative disorders utilizing a radionuclide of lower energy, so that the emitted particle has limited penetration within the cell. Ideally, the particles emitted by the radionuclide would have an average energy low enough that virtually none would escape the cell nucleus, but still possess sufficient energy to cause cell death.

Tritium is a hydrogen isotope having one proton and two neutrons (atomic weight: approx. 3) that emits a low-energy beta particle. The average energy of the emitted beta particle is approximately 0.0055 MeV (Gregory D P and Landsman D A. (1958) “Average Decay Energy of Tritium.” Phys. Rev. 109: 2091-2097), which corresponds to an average penetration in tissue of less than one micron (Caro L G (1962) “High Resolution Autoradiography. II. The Problem of Resolution.” J. Cell. Biol. 15: 189-199). Although tritium has a physical half-life of approximately 12.3 years, it has an effective biological half-life of approximately 12 days (Caro, supra). The average energy of the emitted beta particle is low enough that the majority of beta particles emitted from an intranuclear tritium source would remain within the cell nucleus. However, beta particles emitted from tritium are considered too weak to cause significant DNA damage (Straus et al.

“The Uptake, Excretion, and Radiation Hazards of Tritiated Thymidine in Humans,” Cancer Res. 37: 610-618).

Tritium has been used as a molecular tag in a wide range of in-vivo studies. Tritium has also been used to measure cell proliferation (Meyer J S et al. (1976) “Tritiated Thymidine Labeling Index of Benign and Malignant Human Breast Epithelium.” J. Surg. Oncol. 8:165-181; Denekamp et al. (1973) “In Vitro and In Vivo Labeling of Animal Tumors With Tritiated Thymidine.” Cell. Tissue Kinet. 6: 217-227) or patterns of cell division in normal and tumor tissue (Post et al. (1977) “The Proliferative Patterns of Human Breast Cancer Cells In Vivo.” Cancer 39: 1500-1507; Young et al. (1970) “Cell Cycle Characteristics of Human Solid Tumors In Vivo.” Cell Tissue Kinet. 3: 285-290). The express purpose of these tritium labeling studies was to measure cell proliferation in certain experimental settings. The tritium label was not expected to disturb cell proliferation and confound the results. Thus, while useful as molecular tag, tritium and tritium-labeled compounds have not heretofore been employed as therapeutics for treating cancerous and non-cancerous proliferative disorders.

### SUMMARY OF THE INVENTION

Surprisingly, it has now been found that tritium delivered directly to the cell nuclei of tissues associated with proliferative disorders by the compounds and methods of the present invention is effective in killing cells associated with proliferative disorders. The present invention thus provides methods of treating

b) administering an effective amount of a tritiated nuclear targeting agent to said subject so that the target cells are exposed to the tritiated nuclear targeting agent; and

c) allowing said tritiated nuclear targeting agent to be transported to the target cell nuclei such that the tritiated nuclear targeting agent associates with the target cell DNA,

whereupon the tritiated nuclear targeting agent causes target cell death.

In one embodiment, the tritiated nuclear targeting agent is a steroid hormone. In another embodiment, the agent is an oligonucleobase. In a further embodiment, the agent is a DNA precursor molecule.

In another aspect of the present invention, various modes of administration of the tritiated nuclear targeting agent are provided. In one embodiment, the agent is administered by direct application to the target tissue. Such direct application includes the delivery of the agent by a medical device such as a catheter. In a further embodiment, the agent is administered by direct injection into the target tissue. In another embodiment, the agent is administered systemically to the subject. In yet another embodiment, the agent is administered by repeated systemic injections, repeated direct applications to the target tissue, or repeated injections into the target tissue. In a still further embodiment, the agent is administered in a sustained dose, for example by sustained systemic or subcutaneous infusion over a prolonged period of time.

In another aspect of the present invention, the tritiated nuclear targeting agent is associated with a structure that serves to protect the agent from degradation or clearance by the body. The structure can also serve to direct the agent to the target cell. In one embodiment, the structure is a liposome. In a further embodiment, the liposome is modified so as to avoid clearance by the mononuclear macrophage and reticuloendothelial systems. In a still further embodiment, the liposome carries targeting groups that direct the liposome to the target cells. proliferative disorders utilizing the low-energy radionuclide tritium, which is delivered directly to proliferative tissue cell nuclei.

It is therefore an object of the invention to provide methods of treating proliferative disorders by the delivery of tritium to cell nuclei of tissues associated with the proliferative disorder (e.g., tumors or areas of restenosis). The tissue associated with a proliferative disorder is hereinafter called the “target tissue,” and the cells of tissue associated with a proliferative disorder are hereinafter called “target cells.” Of course, target tissue and target cells may also be described with the name of the particular disorder or tissue; i.e. “tumor cells” or “restenotic tissue.”

Delivery of tritium to target cell nuclei is accomplished by associating tritium with an agent that specifically targets the nucleus of a target cell. Agents that specifically target a target cell nucleus are called “nuclear targeting agents.” Nuclear targeting agents associated with tritium radionuclides are called “tritiated nuclear targeting agents.” Hereinafter, “tritiated nuclear targeting agent” and “agent” are used interchangeably.

It is a further object of this invention to provide methods of treating proliferative disorders by delivering tritiated nuclear targeting agents to target cells with a structure such as a liposome, micelle or microcapsule.

It is a still further object of this invention to provide pharmaceutical formulations comprising tritiated nuclear targeting agents for use in treating proliferative disorders.

These and other objects of the invention will be apparent from the disclosure.

According to one aspect of the present invention, there is provided a method of treating a proliferative disorder comprising the steps of:

a) providing a subject having tissue associated with a proliferative disorder, wherein said tissue comprises target cells having DNA-containing nuclei;

Further embodiments include modified and unmodified micelles and microcapsules associated with a tritiated nuclear targeting agent.

In another aspect of the present invention, a pharmaceutical formulation for treating proliferative disorders is provided comprising a tritiated nuclear targeting agent, wherein the pharmaceutical formulation is at least sterile and pyrogen free.

### BRIEF DESCRIPTION OF THE FIGURES

FIG. 1 shows the in vitro cell survival in percent for three different cancer cell lines (4047 rat colon cancer, and BT-20 human breast cancer and MCF-7 human breast cancer) incubated with tritiated water and tritiated thymidine. In the figure:

-●- MCF7:3H-water

-∘- MCF7:3H-thymidine

-♦- BT20:3H-water

-⋄- BT20:3H-thymidine

-▪- 4047:3H-water

-□-4047:3H-thymidine

FIG. 2 shows the results of the direct injection of 0.4 μCi of ³H-thymidine into human BT-20 breast cancer tumors grown on nude mice. R (the ratio of initial tumor size to final tumor size) is plotted. Twenty animals are represented, comprising 10 animals in each of two groups. Animals are paired together roughly by tumor size at time of treatment.

### DETAILED DESCRIPTION OF THE INVENTION

The present invention provides methods of treating proliferative disorders in a subject by direct delivery of tritium to the nuclei of target cells. A subject can be any animal suffering from a proliferative disorder, including birds, fish, and mammals. The expression “animal” includes human being. It is preferred that the subject is a mammal, for example a rodent (e.g.; mouse, rat, rabbit, guinea pig, etc.) or a human being. Most preferably, the subject is a human being.

As discussed above, a proliferative disorder is characterized by the uncontrolled growth of cells of certain tissue type or types. Such disorders can be classified as cancerous or non-cancerous, depending largely on whether cells associated with the proliferative disorder can evade the normal controls on cell growth and position (cancerous), or whether the cells have evaded only the controls on growth (non-cancerous). Both forms of proliferative disorders may be treated according to the present invention.

Cancerous proliferative disorders are often associated with the growth of tumors. Tumors associated with cancerous proliferative disorders include, but are not limited to: breast, prostate, ovarian, lung, colorectal, brain (i.e, glioma) and renal tumors. Cancerous proliferative disorders may also cause the uncontrolled growth of diffuse malignant cell populations, such as in the leukemias.

Non-cancerous proliferative disorders include, but are not limited to, the following: hemangiomatosis in the newborn, secondary progressive multiple sclerosis, chronic progressive myelodegenerative disease, neurofibromatosis, ganglioneuromatosis, keloid formation, Pagets Disease of the bone, uterine and breast fibrocystic disease, Peronies and Duputren's fibrosis, cirrhosis, atherosclerosis and vascular restenosis.

Vascular restenosis is common in patients being treated for coronary artery disease (CAD) and involves the abnormal proliferation of vascular smooth muscle cells (VSMCs). Restenotic lesions are likely due to a combination of mechanical trauma from surgical procedures used to treat CAD, and ineffective dosing of the lesion with ionizing radiation.

Here, the present methods, compounds and formulations are well suited to controlling non-cancerous proliferative diseases such as VSMC proliferation by direct delivery of tritium to the nuclei of VSMCs. The present methods, compounds and formulations are also well suited to controlling the proliferation of cancerous proliferative diseases, e.g., by direct delivery of tritium to the nuclei of tumor cells.

As discussed above, tritium is a hydrogen isotope having one proton and two neutrons that emits a low-energy beta particle. It has been found that tritium located in the nuclei of target cells emits beta particles that do not escape the cell nucleus, but possess sufficient energy to cause cell death.

Direct delivery of the tritium to a target cell nucleus is accomplished by associated atoms of that isotope with a nuclear targeting agent. Preferably, the nuclear targeting agent associates with or is incorporated into the target cell DNA. Examples of nuclear targeting agents include, for example, steroid hormones, oligonucleobases and nucleic acid precursors.

The steroid hormones are small, hydrophobic molecules derived from cholesterol. If administered to the bloodstream, steroid hormones are transported to target cells by binding reversibly to specific carrier proteins in the blood. Steroid hormones are released from the carrier proteins near a target cell, and diffuse through the target cell membrane to bind reversibly to steroid hormone receptor proteins in the target cell cytosol. The cytosolic hormone/receptor complex has an affinity for DNA that causes these complexes to accumulate in the cell nucleus.

Steroid hormones can also be applied directly to target cells, e.g., by administration via catheter or other placement device, direct injection or subcutaneous infusion near the target tissue site. Steroid hormones applied directly to target cells are taken up by the tumor cells and transported to the target cell nucleus as described above.

Steroid hormones useful in the present invention include, for example, cortisol, estradiol, testosterone, progesterone, tamoxifen and their analogs and derivatives. It is understood that, as used herein, “steroid hormone” includes both agonists and antagonists of naturally occurring steroid hormones. Preferred steroid hormones include those that bind to estrogen receptors expressed in target cells (for example deriving from breast or uterine tissue), and those that bind to testosterone receptors expressed in target cells (for example deriving from prostate or testicular tissue).

An oligonucleobase is a polymer of nucleobases that can hybridize to complementary sequences of target cell DNA. By hybridization, it is meant that complementary oligonucleobases join with the target cell DNA by Watson-Crick base-pairing, i.e., by forming a duplex. It is preferred that oligonucleobases are administered directly to target cells, e.g., by direct injection or subcutaneous infusion near the target tissue. It is particularly preferred that oligonucleobases be associated with a structure such as a liposome, to protect the oligonucleobase from degradation in the body.

The nucleobases comprising an oligonucleobase comprise purine or pyrimidine bases (or a derivative or analog thereof) which are covalently linked to a sugar moiety. Nucleobases include, for example, nucleotides, nucleosides, and nucleotoids. Nucleosides are nucleobases that contain a pentosefuranosyl moiety, e.g., an optionally substituted riboside or 2′-deoxyriboside, and have a linkage to other nucleobases that do not contain a phosphorous atom. Nucleotoids are pentosefuranosyl-containing nucleobases having linkages that contain a phosphorous atom; e.g., phosphorothioates, phosphoroamidates and methylphosphonates. Nucleotides are pentosefuranosyl-containing nucleobases that are linked by phosphodiester groups.

Nucleobases are either of the ribo- or deoxyribo-type. Ribo-type nucleobases contain pentosefuranosyl moieties wherein the 2′ carbon is substituted with a hydroxy, alkyl, or halogen. Deoxyribo-type nucleobases are nucleobases other than ribo-type nucleobases, and include nucleobases which do not contain a pentosefuranosyl moiety.

A preferred oligonucleobase comprises ribonucleotides joined by phosphodiester bonds (RNA). A particularly preferred oligonucleobase comprises deoxyribonucleotides joined by phosphodiester bonds (DNA). Oligonucleobases may be nuclease resistant.

To confer nuclease resistance, oligonucleobases of the invention may contain modified internucleobase linkages, such as phosphorothioate linkages. Thus, the term “oligonucleobase” includes unmodified oligomers of oligonucleobases as well as oligomers wherein one or more purine or pyrimidine moieties, sugar moieties or internucleobase linkages is chemically modified. Preferably, nuclease resistance is conferred on oligonucleobases of the invention by providing nuclease-resistant internucleobase linkages. Many such linkages are known in the art, e.g., phosphorothioate: Zon and Geiser, Anti-Cancer Drug Design, 6:539-568 (1991); Stec et al., U.S. Pat. Nos. 5,151,510; Hirschbein, 5,166,387; Bergot, 5,183,885; phosphorodithioates: Marshall et al, Science, 259:1564-1570 (1993); Caruthers and Nielsen, International application PCT/US89/02293; phosphoramidates, e.g., —OP(═O)(NR¹R²)—O— with R¹ and R² hydrogen or C₁-C₃ alkyl; Jager et al., Biochemistry, 27:7237-7246 (1988); Froehler et al., International application PCT/US90/03138; peptide nucleic acids: Nielsen et al., Anti-Cancer Drug Design, 8: 53-63 (1993), International application PCT/EP92/01220; methylphosphonates: Miller et al., U.S. Pat. Nos. 4,507,433, Ts'o et al., 4,469,863; Miller et al., 4,757,055; and P-chiral link-ages of various types, especially phosphorothioates, Stec et al., European patent application 506,242 (1992) and Lesnikowski, Bioorganic Chemistry, 21:127-155 (1993). Additional nuclease-resistant linkages include phosphoroselenoate, phosphorodiselenoate, phosphoroanilothioate, phosphoranilidate, alkylphosphotriester such as methyl- and ethylphosphotriester, carbonate such as carboxymethyl ester, carbamate, morpholino carbamate, 3′-thioformacetal, silyl such as dialkyl(C₁-C₆)- or diphenylsilyl, sulfamate ester, and the like. Such linkages and methods for introducing them into oligonucleobases are described in many references, e.g., reviewed generally by Peyman and Ulmann, Chemical Reviews 90:543-584 (1990); Milligan et al, J. Med. Chem., 36:1923-1937 (1993); Matteucci et al., International application PCT/US91/06855. The disclosures of all references in this paragraph are herein incorporated by reference in their entirety.

Resistance to nuclease digestion may also be achieved by modifying an internucleotide linkage at both the 5′ and 3′ termini with phosphoroamidites α-cording to the procedure of Dagle et al., Nucl. Acids Res. 18, 4751-4757 (1990), the disclosure of which is incorporated by reference in its entirety.

Some or all of the ribo-type nucleobases of the present oligonucleobases can be nuclease-resistant. Suitable nuclease resistant ribo-type nucleobases can be selected from the group consisting of 2′AX-nucleosides, 2′AX-nucleotoids and 2′AR-nucleotides, where:

A is oxygen or a halogen (preferably fluorine, chlorine or bromine);

X is hydrogen or C₁₋₆ alkyl;

R is C₁₋₆ alkyl; and

when A is a halogen, then X or R is omitted.

Preferred nuclease resistant ribo-type nucleobases are 2′-O methyl ribo-type nucleobases, and particularly preferred are 2′-O methyl ribonucleotides.

Oligonucleobases can be any size polymer which is directed to the cell nucleus. Preferably, any oligonucleobase of at least 2 nucleobases to about 5000 nucleobases may be used in the present invention. More preferably, the oligonucleobase is about 10 to about 40 nucleobases in length. Methods of synthesizing oligonucleobases are known to those skilled in the art.

The nuclcobase sequence of the oligonucleobase targeting agent is not important therapeutically; the sequence is only used to direct the oligonucleobase to the target cell DNA. Preferably, the oligonucleobase is complementary to the coding strand of a specific target cell DNA sequence. Oligonucleobases complementary to the coding strand of the specific target cell DNA sequence will not hybridize with extranuclear RNA derived from that same sequence.

When treating proliferative disorders with an oligonucleobase tritiated nuclear targeting agent, preferred oligonucleobase sequences are those of proto-oncogenes and oncogenes or fragments thereof.

Proto-oncogenes are normal cellular genes, the alteration of which engenders a transforming allele or “oncogene.” Damage to one or more proto-oncogenes has been found in a variety of human malignancies. A large number and variety of human tumors contain consistent point mutations in proto-oncogenes. Chromosomal translocations also contribute to tumorigenesis by activating proto-oncogenes to oncogenes, e.g., the translocation of c-abl to the BCR locus to form the hybrid oncogene bcr-abl which has been correlated with the occurrence of Philadelphia chromosome-positive leukemias. Other tumors carry abnormally amplified domains of DNA that can include proto-oncogenes and magnify their expression (Alitalo & Schwab, Adv. Cancer Res. 47,235-282, 1986). The potential of proto-oncogenes to participate in tumorigenesis arises from the fact that their protein products are relays in the biochemical circuitry that governs the phenotype of vertebrate cells (Bishop, Cell 64, 235-248, 1991).

For example, useful proto-oncogene sequences include, but are in no way limited to, sequences derived from the proto-oncogenes c-myb and c-myc. One such sequence is the c-myc sequence CAC GTT GAG GGG CAT (SEQ ID NO:1). Other c-myc sequences useful in the present invention included SEQ ID NOS: 2-5, 13 and 14 from WO 94/15646 of Thomas Jefferson University, the entire disclosure of which is incorporated by reference in its entirety.

Other suitable sequences include, but are in no way limited to, the following:

| | | |
|---|---|---|
| FOS | GCC CGA GAA CAT CAT | SEQ ID NO:2 |
| JUN | CCT CGC AGT TTC CAT | SEQ ID NO:3 |

One of ordinary skill in the art can readily identify proto-oncogene and oncogene sequences useful in the present invention, and synthesize oligonucleobases corresponding to those sequences. For example, Calabretta et al. provide an extensive list of proto-oncogenes in U.S. Pat. No. 5,734,039, the disclosure of which is herein incorporated by reference in its entirety.

WO 99/34814 of Temple University discloses sequences of the interferon responsive transcript (IRT-1) gene, which are also useful in the present invention. The IRT-1 gene is active in proliferating vascular smooth muscles cells. The disclosure of WO 99/34814 is herein incorporated by reference in its entirety.

Nucleic aid precursors are substances which are incorporated into DNA or RNA by the cell. Nucleic acid precursors include any of the above-mentioned nucleobases which can be incorporated into DNA or RNA by the cell, especially the nucleotide bases. Preferred nucleotide bases include adenosine, cytidine, guanosine, thymidine, and uridine, and analogs and derivatives thereof. Thymidine is preferred because this nucleotide base is only incorporated into DNA. Thus, virtually all tritiated thymidine administered to a subject would deliver the tritium radionuclide to the tumor cell nucleus.

Nucleic acid precursors also include the component molecules of nucleobases, and include, for example, purines or pyrimidines. Purines and pyrimidines include, for example, adenine, guanine, cytosine, thymine and uracil, and their derivatives or analogs.

Nucleic acid precursors are actively taken up by proliferating cells, such as tumor cells, and transported to the cell nucleus for incorporation into DNA or RNA. It is preferred that nucleic acid precursors of the present invention be incorporated into the DNA.

Methods of associating a nuclear targeting agent with tritium will be apparent to those skilled in the art. For example, a nuclear targeting agent can be directly labeled with tritium by the substitution of tritium for a hydrogen on the nuclear targeting agent. Alternatively, a nuclear targeting agent can be synthesized in the presence of tritium so that the tritium is incorporated into the atomic structure of the nuclear targeting agent. As used herein, any substance associated with at least one tritium nucleus is called “tritiated.” A tritiated substance is also designated in this specification by use of the prefix “³H-”, for example as in “³H-thymidine.”

Many tritiated nuclear targeting agents useful in the present invention are commercially available. In particular, tritiated nucleic acid precursors are available from Amersham Pharmacia Biotech, Inc., 800 Centennial Ave., P.O. Box 1327, Piscataway, N.J. 08855 USA, such as ³H-adenosine, ³H-guanosine, ³H-cytidine, ³H-thymidine and ³H-uridine. Tritiated steroid hormones are also available from Amersham Pharmacia Biotech, including ³H-testosterone, ³H-oestradiol, 3H-progesterone, ³H-corticosterone, ³H-dexamethasone and ³H-tamoxifen. A list of available tritiated steroid hormones is given in a table entitled “Selection Guide—Steroid Receptors” on page 84 of the 1999 Amersham Pharmacia Biotech catalog, which table is herein incorporated by reference.

It is apparent that tritiated nucleobases and/or tritiated nucleic acid precursors can be administered directly to subjects as a nuclear targeting agent, or can be used to synthesize oligonucleobases which are then administered to subjects as a nuclear targeting agents. Techniques for synthesizing oligonucleobases from nucleobases or nucleic acid precursors are well-known to those skilled in the art.

The tritiated nuclear targeting agents of the invention (or pharmaceutical formulations of the nuclear targeting agents), can be administered by any method designed to expose target cells to the agent so that the agent is taken up by the target cells and transported to the cell nucleus. Parenteral administration is preferred. For example, suitable parenteral administration methods include intravascular administration (e.g., intravenousbolus injection, intravenous infusion, intra-arterial bolus injection, intra-arterial infusion and catheterinstillation into the vasculature), peri- and intra-target tissue injection (e.g., peri-tumoral and intra-tumoral injection), and direct application to the target tissue, for example by a catheter or other placement device. Suitable parenteral methods also include subcutaneous injection or deposition including subcutaneous infusion (such as by osmotic pumps). It is preferred that subcutaneous injections or infusions be given in the area near the target tissue, particularly if the target tissue is on or near the skin.

Where the administration of the agent is by injection or direct application, the injection can be in a single dose or in multiple doses. Where the administration of the agent is by infusion, the infusion can be a single sustained dose over a prolonged period of time or multiple infusions. Injection of the agent into the target tissue is preferred. Multiple injections of the agent into the target tissue are particularly preferred.

Nucleic acid precursors are generally taken up only by actively growing cells. Thus, to ensure that substantially all target cells take up and incorporate tritiated nucleic acid precursors, it is preferred that the target cells be chronically exposed to this particular type of nuclear targeting agent. Chronic exposure is defined as exposure for a period of time during which the majority of target cells enter the S-phase of the cell cycle (i.e., are actively growing). For agents that do not depend on the cell cycle for uptake, i.e. the steroid hormones and oligonucleobases, it is not critical that tumor cells pass through S-phase. It is particularly preferred that target tissue from cancerous proliferative disorders, for example tumors, be chronically exposed to tritiated nucleic acid precursors.

The period of time during which the majority of target cells enter the S-phase of the cell cycle can be readily determined by one of ordinary skill in the art (see e.g., Tubiana M and Malaise E (1976), Cancer Treatment Reports 60: 1887-1895). As used herein, “majority of target cells” means about 80%, preferably about 90%, and more preferably about 95% or greater of a target cell population has entered the S-phase. A population of target cells includes discreet groupings (e.g., tumors, specific restenotic areas) or the entire number of target cells in a subject.

One way to ensure chronic exposure of target cells to ³H-nucleic acid precursors is by peri- or intra-target tissue injection of a high dose of the agent. For example, a high dose of ³H-thymidine can be injected into a tumor (intra-tumoral injection) or around or near the tumor site (peri-tumoral injection). Injection of a high dose in this manner ensures that effective levels of agent persist in the target tissue for several days. An alternative strategy for chronic exposure involves multiple injections or infusions of agent over time to maintain a sufficient concentration for days or weeks, so that actively cycling target cells will incorporate sufficient agent to cause target cell death. Another strategy for chronic exposure involves the sustained infusion of agent for a period of time during which the majority of target cells enter the S-phase of the cell cycle. The sustained infusion can be, for example, intravascular or subcutaneous.

Tritiated nuclear targeting agents can also be associated with a structure, for example a liposome, micelle or microcapsule, to facilitate the direction of the agent to target cells. The structure can also serve to protect the agent from degradation or clearance by the body. Tritiated nuclear targeting agents associated with structures are administered as described above.

It is preferred that the amount of agent administered to a subject suffering from a proliferative disorder is expressed in terms of the tritium activity. Tritium activity is typically given in terms of microCuries (μCi), but of course can be expressed in any units of radioactivity relevant to disintegration of tritium nuclei. Those skilled in the art are familiar with techniques for measuring the activity of tritium in tritium-labeled compounds. For example, tritium activity can be measured by scintillation counting.

Tritiated nuclear targeting agents, or pharmaceutical formulations thereof, are administered to a subject suffering from a proliferative disorder in any amount effective to cause target cell death. As used herein, an amount of tritiated nuclear targeting agent effective to cause target cell death is any amount which causes a measurable decrease in viable target cells in the subject. An effective amount of tritiated nuclear targeting agent is referred to herein as a “dose.”

Techniques to determine the number of viable target cells in a subject include biochemical and histological techniques for detecting cell death or necrotic tissue. Such biochemical and histological techniques are familiar to those skilled in the art.

Target cell death can also be inferred from a reduction in target tissue size upon treatment with tritiated nuclear targeting agents. Reduction in target tissue size can be ascertained visually or by diagnostic imaging methods including, for example, X-ray, magnetic resonance imaging, ultrasound, and scintigriphy. Diagnostic imaging methods used to ascertain reduction in target tissue size can be employed with or without contrast agents. Such diagnostic imaging methods (both with and without contrast agents) are well known to those of skill in the art.

Reduction in target tissue size can also be ascertained by physical means. Such physical means include, for example, palpation of the target tissue mass, or measurement of the target tissue mass at different times during treatment with a measuring instrument such as a caliper.

A dose of tritiated nuclear targeting agent can be based on the approximate or estimated mass of the target tissue to be treated. Techniques for approximating or estimating target tissue mass are well known in the art. For example, target tissue (e.g., tumor) mass can be estimated by calculating the approximate tissue volume and considering one gram of mass equivalent to one cubic centimeter of tissue volume.

For example, a dose of tritiated nuclear targeting agent based on target tissue mass can be at least about 1 μCi/gram of tumor, and is preferably between about 1-1000 μCi/gram of tumor. More preferably, the dose is at least about 60 μCi/gram of target tissue. Particularly preferably, the dose is at least about 100 μCi/gram of target tissue. It is preferred that doses of tritiated nuclear targeting agent based on target tissue mass be injected directly into the target tissue. However, doses based on target tissue mass can also be administered systemically (e.g., intravascularly, subcutaneously, intramuscularly or intraperitoneally) or by direct application to the target tissue.

A dose of tritiated nuclear targeting agent can also be based on the approximate or estimated body weight of the subject to be treated. Preferably, body weight doses are used for systemic administrations including, for example, intravascular injections and infusions, subcutaneous depositions or infusions, and intramuscular or intraperitoneal administrations.

For example, single injection intravascular doses in humans (assuming a 60 kg subject) can range from about 5-3000 μCi/kg of body weight, and are preferably between about 700-1000 μCi/kg of body weight. Such doses are more preferably greater than about 1000 μCi/kg of body weight. The same doses for single intravascular injections can also be used for multiple intravascular injections, although for multiple injections the dose may also be lower. For example, multiple intravascular injection doses in humans are preferably greater than about 250 μCi/kg body weight, and particularly preferably greater than about 500 μCi/kg body weight.

Single sustained infusion intravascular doses can be the same as those used for single and multiple intravascular injections, but may also be lower. For example, doses for single sustained infusions are preferably greater than about 90 μCi/kg body weight, and more preferably are greater than about 100 μCi/kg.

Multiple sustained infusion intravascular doses can be the same as those used for single and multiple injection and single sustained intravascular doses, but may also be lower. For example, doses for multiple sustained intravascular infusions are preferably greater than about 35 μCi/kg, and more preferably are greater than about 50 μCi/kg.

Subcutaneous, intramuscular and intraperitoneal doses can be the same as for the intravascular doses but are preferably between about 200 and 1000 μCi/kg body weight. More preferably, such doses are greater than 500 μCi/kg of body weight.

A dose of tritiated nuclear targeting agent can further be based on the approximate or estimated surface area of the subject to be treated. Surface area doses for a given subject are typically expressed in terms of μCi tritiated nuclear targeting agent/square meter of surface area (m²). It is preferred to base doses of a tritiated nuclear targeting agent on the surface area of a subject, because better inter-species dose comparisons can be made. Also, doses based on surface area allow doses to be determined for human adults and children without further adjustment. The assumptions underlying the inter-species and adult to child conversion of doses based on surface area are found in E J Freireich et al., (1966) “Quantitative comparison of toxicity of anticancer agents in mouse, rat, dog, monkey and man,” Cancer Chemotherapy Reports 50: 219-244, the disclosure of which is herein incorporated by reference in its entirety.

Table 1 provides approximate surface area to weight ratios for various species. The surface area to weight ratio can be used to convert body weight doses expressed in terms of μCi/kg to surface area doses. The surface area to weight ratio is also used to calculate the conversion factors found in Table 2. The conversion factors in Table 2 can be used to convert doses expressed in terms of μCi/kg from one species to another.

In Table 1, to express a μCi/kg in any given species as the equivalent μCi/m² dose, multiply the dose by the approximate surface area to weight ratio. For example, in the adult human 100 μCi/kg is equivalent to 100 μCi/kg X 37 kg/m²=3700 μCi/m². Adapted from DeVita, V T, “Principles of Chemotherapy,” pgs. 292-3, in Cancer: Principles and Practice of Oncology, (3^{rd} edit., DeVita V T, Hellman S, and Rosenberg S A, eds.), 1989, J. B. Lipincott Co., Phila., Pa.

TABLE 1
Surface Area to Weight Ratios of Various Species

| | | | Surface Area to |
|---|---|---|---|
| Species | Body Weight (kg) | Surface Area (m²) | Weight Ratio (kg/m²) |
| Mouse | 0.02 | 0.0066 | 3.0 |
| Rat | 0.15 | 0.025 | 5.9 |
| Monkey | 3 | 0.24 | 12 |
| Dog | 8 | 0.40 | 20 |
| Human | | | |
| child | 20 | 0.80 | 25 |
| adult | 60 | 1.6 | 37 |

It is preferred that doses based on surface area be administered systemically, as describe above for doses based on body weight. However, surface area doses can be administered by peri- or intra-target tissue injection or by direct application to the target tissue.

Table 2 gives approximate factors for converting doses expressed in terms of μCi/kg from one species to an equivalent surface area dose expressed in the same units (μCi/kg) in another species. For example, given a dose of 50 μCi/kg in the mouse, the appropriate dose in man (assuming the equivalency on the basis of μCi/m²) is 50 μCi/kg×1/12=4.1 μCi/kg. For the present invention, equivalency on the basis of is μCi/m² assumed. Adapted from DeVita, V T, “Principles of Chemotherapy,” pgs. 292-3, in Cancer: Principles and Practice of Oncology, (3^{rd} edit., DeVita V T, Hellman S, and Rosenberg S A, eds.), 1989, J. B. Lipincott Co., Phila., Pa.

TABLE 2
Equivalent Surface Area Dosage Conversion Factor

| | Mouse | Rat | Monkey | Dog | Man |
|---|---|---|---|---|---|
| | (20 g) | (150 g) | (3 kg) | (8 kg) | (60 kg) |
| Mouse | 1 | 1/2 | 1/4 | 1/6 | 1/12 |
| Rat | 2 | 1 | 1/2 | 1/4 | 1/7 |
| Monkey | 4 | 2 | 1 | 3/5 | 1/3 |
| Dog | 6 | 4 | 5/3 | 1 | 1/2 |
| Man | 12 | 7 | 3 | 2 | 1 |

The present invention also provides pharmaceutical formulations comprising the tritiated nuclear targeting reagents. Pharmaceutical formulations of the present invention are characterized as being at least sterile and pyrogen-free. As used herein, “pharmaceutical formulations” include formulations for human and veterinary use.

Examples of pharmaceutical formulations include agents mixed with a physiologically acceptable carrier medium to form solutions, suspensions or dispersions. Preferred physiologically acceptable carrier media are water or normal saline. Pharmaceutical formulations can also include conventional pharmaceutical excipients and/or additives. Suitable pharmaceutical excipients include, for example, stabilizers, antioxidants, osmolality adjusting agents, buffers, and pH adjusting agents. Suitable additives include, for example, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA, CaNaDTPA-bisamide), or, optionally, additions (e.g., 1 to 50 mole percent) of calcium or sodium salts (for example, calcium chloride, calcium ascorbate, calcium gluconate or calcium lactate). Pharmaceutical formulations according to the present invention can be prepared in a manner fully within the skill of the art.

In a further embodiment of the present invention, the tritiated nuclear targeting agent is carried by a structure. Examples of structures useful for carrying an agent include liposomes, micelles, and microcapsules. The structure can serve to more effectively direct the agent to target cells. The structure can also serve to protect the agent from degradation or clearance by the body. Structures useful in the present invention can be modified to affect their biodistribution, for example by having opsonization inhibition moieties or targeting groups bound to the surface of the structure. In a preferred embodiment, a structure has both opsonization inhibition moieties and targeting groups bound to its surface.

In one embodiment, the structure is a liposome. As used herein, “liposome” refers to a generally spherical entity formulated from amphiphilic compounds which is characterized by the presence of at least one internal void. Preferred amphiphilic compounds are lipids. In any given liposome, the amphiphilic compounds may be in the form of a one or more monolayers or bilayers. Where a liposome comprises more than one mono- or bilayer, the mono- or bilayers are generally concentric. The liposomes described herein include such entities commonly referred to as liposomes, bubbles, microbubbles, microspheres, vesicles and the like. Thus, the amphiphilic compounds may be used to form a unilamellar liposome (comprised of one monolayer or bilayer), an oligolamellar liposome (comprised of about two or about three monolayers or bilayers) or a multilamellar liposome (comprised of more than about three monolayers or bilayers). As used herein, the term “liposome” also refers to multivesicular liposomes, which are liposomes comprising multiple non-concentric voids. For examples of multivesicular liposomes and methods of their preparation, see U.S. Pat. Nos. 5,993,850 of Sankaram et al. and 5,997,899 of Ye et al., the disclosures of which are herein incorporated by reference in their entirety. Multivesicular liposomes are especially useful for sustained or timed release of the tritiated nuclear targeting agents.

The internal voids of the liposomes may be filled with a liquid, including, for example, an aqueous liquid, a gas, a gaseous precursor, and/or a solid or solute material.

In one aspect of the present invention, tritiated nuclear targeting agents are carried by liposomes. As used herein, “carried by liposomes” means the agent is embedded within the wall of the liposome, encapsulated in the liposome or attached to the liposome. As used herein, “attached to” means that the agent is associated in some manner to the inside and/or the outside wall of the liposome, such as through a covalent or ionic bond, or other means of chemical or electrochemical linkage or interaction. As used herein, “encapsulated in” means that the agent is located in the internal liposome void. As used herein, “embedded within” means the agent is within the liposome wall. Thus, the agent can be positioned variably, such as, for example, entrapped within the internal void of the liposome, incorporated onto the internal/external surfaces of the liposome and/or enmeshed within the liposome structure itself.

Preparation of liposomes carrying tritiated nuclear targeting agents is within the skill of those in the art. Where the agent is lipophilic or amphiphilic, efficient embedding within the liposome wall can be achieved by preparing a mixture of liposome-forming material and the agent, e.g., in a dried film, and hydrating the mixture. This procedure will form liposomes with lipophilic or amphiphilic agent embedded predominantly in the wall of the liposomes. Useful techniques for incorporating amphiphilic compounds into liposome membranes are disclosed, e.g., in Grant et al., Magn. Res. Med., 11:236-243 (1989); Kabalka et al., Magn. Res. Med., 8:89-95 (1988); and Hnatowich et al., J. Nucl. Med., 22:810-816 (1981), the disclosures of which are herein incorporated by reference in their entirety.

Alternatively, the amphiphilic material used in forming the liposomes can be conjugated with the agent prior to liposome formation. Liposomes formed with amphiphilic material conjugated with the agent will carry the agent attached to both the inner and outer liposome surfaces. The agent can also be conjugated to the liposome after it has been formed, which will result in the agent being carried only on the outside surface of the liposome.

Passive loading may also be employed for preparing liposomes with encapsulated hydrophilic trititated nuclear targeting agents. In this case, the hydrophilic agent is usually dissolved in the aqueous medium used to hydrate a film of liposome-forming material. Typically, the aqueous medium containing the film is sonicated to form liposomes encapsulating the agent dissolved in the aqueous solution. Depending on the hydration conditions and the nature of the agent, encapsulation efficiencies of between about 5-20% are typically obtained, with the remainder of the agent being in the bulk aqueous phase. An additional processing step for removing non-encapsulated agent is therefore usually required. For other techniques by which water-soluble materials are encapsulated in liposomes; see e.g., Bangham et al. J. Mol. Biol., 13:238-252 (1965); D. Papahadjopoulos and N. Miller. Biochim. Biophys. Acta, 135:624-638 (1967); Batzri and Korn. Biochim. Biophys. Acta, 2981015 (1973); Deamer and Bangham. Biochim. Biophys. Acta, 443:629-634 (1976); Papahadjopoulos et al. Biochim. Biophys. Acta, 394:483491 (1975); German Pat. No. 2,532,317; and U.S. Pat. Nos. 3,804,776; 4,016,100 and 4,235,871, the disclosures of which are herein incorporated by reference in their entirety.

A more efficient method for encapsulating hydrophilic compounds, involving reverse evaporation from an organic solvent, has been reported; see Szoka, F., Jr., et al., (1980) Ann. Rev. Biophys. Bioeng. 9:467, the disclosure of which is herein incorporated by reference in its entirety. In this approach, a mixture of hydrophilic agent and liposome-forming lipids are emulsified in a water-in-oil emulsion, followed by solvent removal to form an unstable lipid-monolayer gel. When the gel is agitated, typically in the presence of added aqueous phase, the gel collapses to form oligolamellar liposomes with high (up to 50%) encapsulation of the agent.

In the case of ionizable hydrophilic or amphipathic trititated nuclear targeting agents, even greater agent-loading efficiency can be achieved by loading the agent into liposomes against a transmembrane ion gradient, see Nichols, J. W., et al., Biochim. Biophys. Acta 455:269-271 (1976); Cramer, J., et al., Biochemical and Biophysical Research Communications 75(2):295-301 (1977), the disclosures of which are herein incorporated by reference in their entirety. This loading method, generally referred to as remote loading, typically involves an agent having an ionizable amine group which is loaded by adding it to a suspension of liposomes prepared to have a lower inside to higher outside ion gradient, for example a pH gradient.

The liposomes of the present invention deliver the carried agent at or near the target cells. The liposomes can fuse with a target cell, be taken up by a target cell, or release their contents outside a target cell. Regardless, the agent will be delivered to the target cell nucleus. Liposomes are particularly effective at delivering agents to tumor cells. Suitable doses of tritiated nuclear targeting agents carried by liposomes are as disclosed above.

The liposomes of the present invention can be any size. Particularly preferred liposomes are those which are small enough to pass through the pulmonary capillary bed; i.e., those with a diameter of approximately 8 microns. However, liposomes useful in the present invention can have a diameter of about 0.1 to about 2,000 microns. Preferred liposomes are those having a diameter of about 100 to 1,000 microns, others having a diameter of about 10 to 100 microns, and still others having a diameter of about 1 to 100 microns. A preparation of liposomes typically has a distribution of sizes. A liposome preparation in a range of about 0.1 to 10 microns with a size distribution within less than about a 20% standard deviation of the average diameter is preferred. A liposome preparation in a range of about 0.1 to 10 microns with a size distribution within about 10% of the average diameter is still more preferred.

Delivery of an agent may be enhanced by the external application of energy to the delivery site, for example by the application of ultrasound to the tumor site. The ultrasound energy will either enhance the fusion of the liposomes with the target cell, or cause the liposomes to break, thus exposing target cells to the trititated nuclear targeting agent.

Preferred liposomes are “modified liposomes.” Modified liposomes carry components on their outer surface that affect biodistribution, for example opsonization inhibiting moieties or targeting groups with a specific affinity for a target cell. A modified liposome can comprise opsonization inhibiting moieties and targeting groups.

Opsonization-inhibiting moieties are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization inhibiting hydrophilic polymers form a protective surface layer which significantly decreases the uptake of the liposomes by the macrophage-monocyte system (MMS) and reticuloendothelial system (RES), e.g., as described in U.S. Pat. No. 4,920,016, which is herein incorporated by reference in its entirety. Liposomes modified with opsonization inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example solid tumors, will efficiently accumulate these liposomes; see Gabizon, et al., P.N.A.S., USA, 18:6949-53 (1988). In addition, the reduced uptake by the RES lowers the toxicity of stealth liposomes carrying tritiated nuclear targeting agent by preventing significant accumulation in the liver and spleen. Thus, liposomes that are modified with opsonization inhibition moieties deliver the agent to tumor cells, whereupon the agent will be transported to the target cell nucleus.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives, e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM₁. Copolymers of PEG, methoxy PEG, or methoxy PPG, or derivatives thereof, are also suitable. In addition, the opsonization inhibiting polymer may be a block copolymer of PEG and either a polyamino acid, polysaccharide, polyamidoamine, polyethyleneamine, or polynucleotide. The opsonization inhibiting polymers may also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

The opsonization inhibiting polymer can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture such as tetrahydrofuran and water in a 30:12 ratio at 60° C.

To obtain a liposome that is targeted for a specific target cell, a targeting group is bound to the outer surface of the liposome. As used herein, a targeting group is “bound” to a liposome membrane when it is chemically or physically attached to the membrane, e.g., by the intercalation of a lipid-soluble anchor into the membrane itself, or by binding directly to active groups of membrane lipids.

For example, the carbohydrate portion of the liposome membrane is oxidized, e.g., by exposure to sodium metaperiodate to yield aldehyde groups, which are highly reactive and will bind the target group to the membrane. In addition, the target group can be linked to a lipid-soluble anchor, and the anchor is then intercalated into the liposome membrane. These and other methods of binding targeting groups to liposome membranes are described in U.S. Pat. No. 4,483,929, the disclosure of which is herein incorporated by reference in its entirety.

Suitable targeting groups include compounds selected or designed to target a target cell; for example polyclonal or monoclonal antibodies, fragments of antibodies, chimeric antibodies, an enzyme or enzyme substrate, a lectin, a saccharide ligand of a lectin, and small molecule ligands.

A preferred small molecule ligand is the E. coli heat stable enterotoxin ST, which specifically targets cells of colorectal origin, such as metastasized colorectal cancer cells. The E. coli heat stable enterotoxin ST is described in Waldman, U.S. Pat. No. 5,518,888, the disclosure of which is herein incorporated by reference in its entirety.

Preferred antibodies include antibodies to tumor-associated antigens. Specific examples include, for example, B72.3 antibodies (described in U.S. Pat. Nos. 4,522,918 and 4,612,282) which recognize colorectal tumors, 9.2.27 anti-melanoma antibodies, D612 antibodies which recognize colorectal tumors, UJ13A antibodies which recognize small cell lung carcinomas, NRLU-10 (Tfs-2) antibodies which recognize small cell lung carcinomas and colorectal tumors, 7E11C5 antibodies which recognize prostate tumors, CC49 antibodies which recognize colorectal tumors, TNT antibodies which recognize necrotic tissue, PR1A3 antibodies, which recognize colon carcinoma (see Richman, P. I. and Bodmer, W. F. (1987) Int. J. Cancer Vol. 39, pp. 317-328), ING-1 and other genetically engineered antibodies, which are described in WO-A-90/02569, B 174 antibodies (developed at Biomira, Inc. of Edmonton, Canada), which recognize squamous cell carcinomas, B43 antibodies which are reactive with certain lymphomas and leukemias, and other antibodies which may be of particular interest. All references cited in this paragraph are herein incorporated by reference in their entirety.

As many solid tumors are surrounded by an area of neovascularization, other suitable targeting groups include antibodies directed to cell surface antigens of neovascular endothelium (e.g., anti-CD105 antibodies) or ligands with affinity for cell surface receptors of neovascular endothelium.

Also preferred are antibodies directed to markers of vascular restenosis, for example cell surface antigens of vascular smooth muscle cells (VSMCs). VSMCs are a major component of restenotic lesions seen in patients undergoing treatment for coronary artery disease. Other useful antibodies include those directed to the product of the IRT-1 gene, as described in WO 99/34814 supra.

Pharmaceutical formulations of liposomes carrying tritiated nuclear targeting agents may be formulated as described above, but may contain additional emulsifiers and/or viscosity modifiers designed to keep the liposomes in suspension. Suitable viscosity modifiers include, for example, carrageenan, cellulose, dextrin, gelatin, guar gum, hydroxyethyl cellulose, hydroxypropyl methylcellulose, magnesium aluminum silicate, methylcellulose, pectin, polyethylene oxide, polyvinyl alcohol, propylene glycol alginate, silicon dioxide, sodium alginate, tragacanth, and xanthan gum. Suitable emulsifiers include, for example, poloxamers and their derivatives, polyoxyethylene 50 stearate, polyoxyl 35 castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan mono-laurate, sorbitan mono-oleate, sorbitan mono-palmitate, sorbitan monostearate, stearic acid, and emulsifying wax. Pharmaceutical formulations according to the present invention comprising liposomes can be prepared in a manner fully within the skill of the art.

In a further embodiment, the structure is a micelle. A micelle is formed by the spontaneous organization of amphiphilic materials in solution into particles with a hydrophobic core and a hydrophilic corona. A micelle has little or no internal void. Therefore, micelles generally only carry tritiated nuclear targeting agents as associated with the surface of the micelle or as part of the micellar structure. A micelle can also carry agent entrapped within the micellar structure.

Tritiated nuclear targeting agents carried by micelles are preferably amphiphilic or are linked to a lipophilic anchor such that they are incorporated into the micellar structure. However, hydrophobic agents can be carried within a micelle's hydrophilic core. Likewise, a hydrophilic agent can be carried within a micelle's hydrophilic corona. Techniques for constructing micelles and for incorporating materials into micelles or onto micellar surfaces are well known in the art; see for example Torchilin V P, (1997) QJ Nuclear Med. 41: 141-153; Weissig V et al., (1998) Pharm. Res. 15: 1552-1556; Gabizon et al., P.N.A.S., USA, 18:6949-53 (1988), the disclosures of which are herein incorporated by reference in their entirety.

Micelles can also be modified to affect their biodistribution, for example by association with opsonization inhibitors or targeting groups. A surface modifier can be associated with a micelle by attachment (e.g., covalent or ionic bond, or other means of chemical or electrochemical linkage or interaction) to the micellar surface, or by incorporation of the surface modifier into the micellar structure. For example, a long-chain hydrophilic polymer or a targeting group can be conjugated to a lipophilic anchor and assembled into the micellar structure along with the other amphiphilic material. Examples of opsonization inhibiting moieties and targeting groups useful for modifying micelles are as described for liposomes above. Techniques for modifying micelles are well known in the art, see e.g., Torchilin V P, (1997) QJ Nuclear Med. 41: 141-153; Weissig V et al., (1998) Pharm. Res. 15: 1552-1556; Gabizon et al., P.N.A.S., USA, 18:6949-53 (1988), supra.

Micelles are particularly useful in delivering the agents to tumors residing in the lymphatic system, especially when administered by subcutaneous injection or infusion. Micelles modified with opsonization inhibiting moieties are useful in delivering agent to solid tumors when administered intravascularly, as they will accumulate in tissue fed by porous or leaky microvasculature (see Gabizon et al., supra).

A preparation of micelles typically has a distribution of sizes. A micelle preparation a size distribution within less than about a 20% standard deviation of the average diameter is preferred. A micelle preparation in a with a size distribution within about 10% of the average diameter is still more preferred. Preferably, the micelles of the invention are between about 5 nanometers and about 50 nanometers in diameter.

Pharmaceutical formulations of micelles can be prepared as described above for liposomes, using techniques well known to those of skill in the art.

In a still further embodiment, the structure is a microcapsule. Microcapsules are fine dispersions of solids or droplets of liquid onto which a thin film coating has been applied. The average diameter of microcapsules may vary from one micron to several hundred microns depending on the materials used and their method of production. The coating of microcapsules comprises an non-amphiphilic organic polymer, including for example amines (e.g., mono-, di-, tri-, tetra-, and higher amines, mixtures thereof, and mixtures thereof with monoamines), alginic acid, arabic acid, cellulose sulfate, carboxymethylcellulose, carrageenans, chondroitin sulfate, heparin, polyacrylic acid, polyoxyethylene cross-linked polyacrylic acid, polyphosphazine, glycolic acid esters of polyphosphazine, lactic acid esters of polyphosphazine, hyaluronic acid, polygalacturonic acid, polyphenylene sulfonic acid, and polyvinylcarboxylic acid, polymerizable aldehydes, derivatives thereof and mixtures thereof. Examples of microcapsules suitable for use in the present invention are found in U.S. Pat. No. 5,686,113 of Speaker et al., U.S. Pat. No. 5,501,863 of Rossling et al. and U.S. Pat. No. 5,993,374 of Kick, the disclosures of which are incorporated herein by reference in their entirety.

Microcapsules can optionally be modified to affect their biodistribution, for example to alter the microcapsule biodistribution by addition of targeting groups or opsonization-inhibiting moieties. Microcapsules modified in this way have similar characteristics and advantages as modified liposomes and micelles. Opsonization inhibiting moieties and targeting groups useful in surface-modifying microcapsules are as described for liposomes and micelles above. Techniques for preparing and modifying microcapsules are well known in the art (see, for example, U.S. Pat. No. 5,686,113, U.S. Pat. Nos. 5,501,863, and 5,993,374, supra).

The microcapsules of the invention can be any size. Particularly preferred microcapsules are those which are small enough to pass through the pulmonary capillary bed; i.e. those with an diameter of approximately 8 microns. However, microcapsules useful in the present invention can have a diameter of about 0.1 to about 2,000 microns. Preferred microcapsules are those having a diameter of about 100 to 1,000 microns, others having a diameter of about 10 to 100 microns, and still others having a diameter of about 1 to 100 microns. A preparation of microcapsules typically has a distribution of sizes. A microcapsule preparation in a range of about 0.1 to 10 microns with a size distribution within less than about a 20% standard deviation of the average diameter is preferred. A microcapsule preparation in a range of about 0.1 to 10 microns with a size distribution within about 10% of the average diameter is still more preferred.

Pharmaceutical formulations of microcapsules can be prepared as described above for liposomes and micelles, using techniques well known to those of skill in the art (see, for example, U.S. Pat. No. 5,501,863, supra).

In one embodiment of the invention, the tritiated nuclear targeting agents are used to treat tumors.

In another embodiment of the invention, the agents are used to treat restenotic lesions occurring in patients being treated for coronary artery disease.

Clinicians have used mechanical devices (e.g., stent, atherectomy, laser, rotablator, etc.) to physically remove restenotic plaques, including the proliferating vascular smooth muscle cells (VSMCs), which occur after coronary angioplasty.

Particularly preferred methods of administering the present agents to patients suffering from restenosis are intravascular injection or infusion, and direct application to the restenotic lesion. It is preferred that intravascularly administered agents are carried in a structure modified with targeting groups which direct the structure to VSMCs. For example, the agent can be carried in a liposome, micelle or microcapsule which is optionally modified (e.g., with an opsonization inhibition moiety and/or with a targeting group comprising an anti-VSMC antibody). Determination of dosage amounts and dosage regimens is as described above.

Direct application of agent to the restenotic lesion can be accomplished by any device capable of reaching the lesion, such as a catheter designed to deliver a therapeutic drug solution. For example, U.S. Pat. No. 5,087,244 to Wolinsky et al. discloses a catheter having a perforated inflatable balloon for expressing drug to the vascular wall. U.S. Pat. No. 5,021,044 to Sharkawy discloses an infusion catheter having a plurality of effluent flow ports along its outer wall, each having a successively larger diameter in the distal direction. U.S. Pat. No. 4,968,307 to Dake et al. discloses another catheter for infusion of therapeutic fluids, in which each effluent flow port through the wall of the catheter is placed in fluid communication with a fluid source by a unique flow passageway extending throughout the length of and within the wall of the catheter body. U.S. Pat. No. 6,027,487 to Crocker discloses a low profile infusion catheter for delivering thrombolytic drugs to a preselected site, with improved flexibility characteristics and relatively uniform delivery over a preselected axial length. Other useful catheters include the perforated or porous balloon catheters as described in Wolinsky et al. (1990) J. Am. Coll. Cardiol. 15: 475-481 and U.S. Pat. No. 5,993,374 to Kick. The disclosures of all references cited in this paragraph are herein incorporated by reference in their entirety. Use of such catheters to deliver the present agent to restenotic lesions is well known to those of skill in the art.

The invention will be illustrated by the following non-limiting examples.

### EXAMPLE 1

### Killing of Tumor Cells in vitro with ³H-thymidine

In-vitro experiments were performed using cultured 4047 colon cancer cells as described in Gatenby, R A and Taylor D D (1990) Cancer Res. 50: 7997-8001. 4047 cells (2×10⁴) were placed in each well of a 6-well plate and incubated overnight in 1 ml of RPMI-1640 media with 10% fetal bovine serum (FBS). The cells were divided into 4 groups as follows:

Group 1: control

Group 2: incubated with ³H-water (New England Nuclear Life Sciences Products, Boston, Mass.) at final concentrations of 0.1, 0.3, 0.5, 0.7, 0.9, 1.0, 1.5, 1.8, 2.0, and 3.0 μCi/ml media

Group 3: incubated in ³H-thymidine (specific activity 89.9 Ci/mmol; New England Nuclear Life Sciences Products, Boston, Mass.) at final concentrations of 0.1, 0.3, 0.5, 0.7, 0.9, 1.0, 1.5, 2.0, 2.5, 3.0 μCi/ml media

Group 4: incubated in unlabelled thymidine at concentrations 1, 2, 3, and 4 mM (so that concentration of thymidine was identical to that of the labeled thymidine in Group 3).

Each group was incubated for 24 hours and then washed 3 times in sterile physiologic saline (PBS). One milliliter of unlabelled media was added and the cells incubated for an additional 48 hours. Subsequently, the cells were washed with PBS, lifted with trypsin, and counted with a hematocytometer. Percent cell viability was determined by exclusion of trypan blue. All measurements were performed in triplicate. Once a dose-response curve was established in the 4047 cell line, identical experiments were conducted with the BT-20 and MCF-7 breast cancer cell lines. However, in these experiments, concentrations of ³H-water and ³H-thymidine were limited to 1, 1.5, 2.0,2.5, and 3.0 μCi/ml of media.

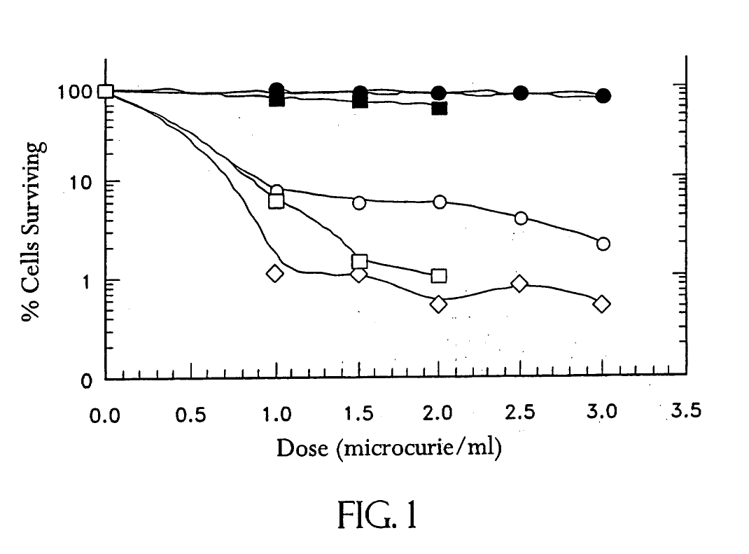

The results of the in vitro experiments are shown in FIG. 1 (results from unlabeled thymidine not shown). The control tumor cell populations grew exponentially in culture. Unlabelled thymidine had no effect on tumor growth. ³H-water delivered radiation uniformly throughout the cell volume and showed slight cytotoxicity, particularly in doses above 1 μCi/ml. However, when the same radiation dose was delivered entirely to the nucleus using ³H-thymidine, significantly greater cytotoxicity was seen at each dose (p<<0.01 by t-test). A significant antitumor effect was seen even at 0.1 μCi/ml. In most experiments, few viable tumor cells could be identified in populations exposed to 1.0 μCi/ml or greater. Thus, it is apparent that delivery of tritium directly to the tumor cell nucleus was at least 100-fold more cytotoxic than the same dose distributed throughout the cell volume.

### EXAMPLE 2

### In vivo Treatment of Human Tumors in a Nude Mouse Model

For consistency with the in vitro research, the BT-20 breast cancer tumor model in nude mice was used. Tumor cells (5×10⁶) were inoculated in the subcutaneous tissue on the flanks of 20 4-6 week old Nu/Nu female nude mice (Charles River Laboratory, Wilmington, Mass.). The mice were weighed and their physical condition was visually monitored every other day. Approximate tumor volumes were measured every other day using calipers to measure the height (H), width (W), and length (L) of each tumor. Tumor volumes were calculated according to the formula for volume of an ellipsoid V=πHWL/6. Tumors grew to 100-200 mm³ in two weeks after inoculation.

The mice were separated into two groups of 10 each. One group received a dose of 0.4 μCi of ³H-thymidine (specific activity 89.9 Ci/mmol; New England Nuclear Life Sciences Products, Boston, Mass.) in 20 μl of saline injected directly into the tumor. The other group received 20 μl of saline injected directly into the tumor. Tumor growth was monitored and recorded daily for a period of 3 weeks.

Over the course of the experiment, no systemic ill effects were observed in either the experimental or control groups of mice. All mice continued to gain weight and there were no apparent behavioral aberrations. Tumor measurements were normalized for variations in initial volume by calculating the results as:

R=S1/S2

where R is the ratio of initial tumor volume (S1) to tumor volume three weeks after treatment (S2). Thus, if no growth occurred, the ratio R would be 1. For tumor remission R would be larger than 1, and for tumor growth R would be less than 1.

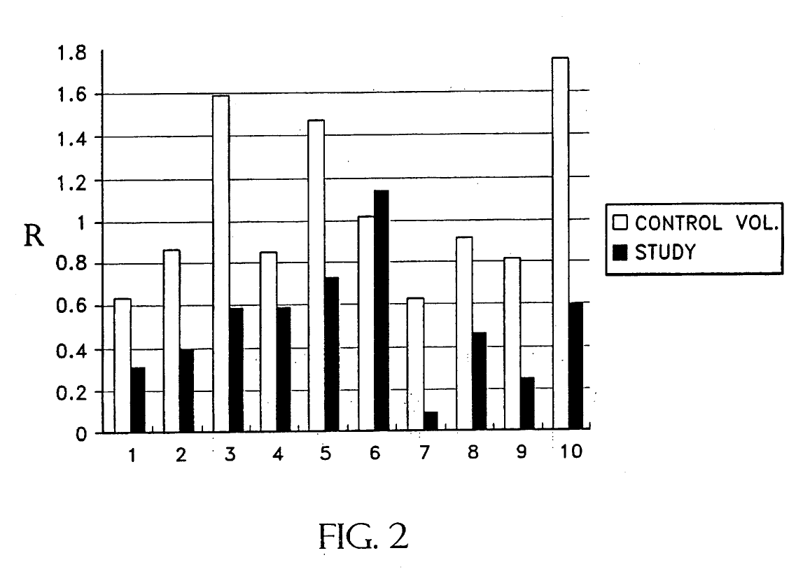

The results of the in vivo experiments are shown in FIG. 2. For the group of mice treated with ³H-thymidine, R=1.201+/−0.49 (indicating tumor remission), while in the control group R=0.58+/−0.48 (indicating tumor growth). This result is statistically significant by t-test at the level of p<0.02. Thus, a statistically significant reduction in tumor size was seen with only one intratumoral injection of 0.4 μCi ³H-thymidine (i.e. 2-4 μCi/gram of tumor).

### EXAMPLE 3

### Biodistribution of ³H-thymidine Following a Single Intravenous or Intratumoral Injection in Tumor-Bearing Mice Over Five Days Post Injection

BT-20 breast cancer tumors are grown in the flanks of nude mice as in Example 2 except that the tumors are grown to an approximate volume of 500 mm³. At time zero, the mice are randomly divided into two groups. The first group receives an intravenous injection of 10 μCi of ³H-thymidine diluted in 20 μl of normal saline for an approximate dose of 0.5 μCi per gram of body mass. The second group receives 10 μCi of ³H-thymidine diluted in 20 μl of normal saline injected into the center of the tumor. Ten animals from each group are sacrificed at 1 hour, 24 hours, 48 hours, 96 hours, and 120 hours following administration of ³H-thymidine. Levels of ³H-thymidine are determined for each animal in blood, bone marrow, liver, spleen, kidney and small intestines since these represent the major sites of ³H-thymidine incorporation following systemic administration. To measure the distribution and retention of ³H-thymidine in the tumors, each tumor is divided into 8 sections by bisecting the lesion three times. The level of ³H-thymidine is determined separately for each section.

Each sample of normal or tumor tissue is weighed and the level of tritium determined using methodology described by Larson et al. (1981) J. Nucl. Med. 22(10) 869-874, the entire disclosure of which is herein incorporated by reference. Each sample is oxidized using an automatic sample oxidizer. Tracer content is determined using a liquid scintillation counter.

The Larson et al. method may overestimate the amount of ³H-thymidine present in the cell nuclei since some of the tritium presumably detaches from the thymidine and some of the tritiated thymidine may be metabolized. To determine the extent of “free” tritium, additional analyses are performed on the blood and tumor samples from two of the animals in each group. These samples are lysed and fractionated using HPLC. The presence of tritium in each fraction is determined using a liquid scintillation counter. Any sample that has tritium present is further analyzed with reverse phase HPLC and compared to the retention times for water, thymidine, and thymine (the major thymidine metabolite) standards.

Slight variations in the above described experimental design are not expected to significantly change the anticipated results.

Anticipated Results—It is expected that, after systemic injection, about 5 to 10% of the ³H-thymidine will be found in tumor tissue, and about 60 to 70% will be found in the intestine and bone marrow. The rest will likely be found in blood, liver and kidneys. Approximately 50% of the thymidine should be excreted in 5 days (˜120 hours). Trace amounts of ³H-water and ³H-thymidine are expected to be found in the liver and blood throughout the experiment.

After intratumoral injection, it is expected that about 50% of the injected ³H-thymidine dose will be retained in the tumor at one hour. The ³H-thymidine concentration will be reduced by about 50% by approximately 120 hours. Only about 10% of the administered dose is expected to be found in the bone marrow and intestine. Significant concentrations of ³H-thymidine are expected to be found in the blood and liver at 1 hour and 24 hours.

### EXAMPLE 4

### Biodistribution of ³H-thymidine Following Multiple Intratumoral Injections in Tumor-Bearing Mice at Four Weeks Post-Injection

This experiment is designed to measure tumor growth, systemic toxicity, concentration and distribution of intratumorally injected ³H-thymidine in tumor, liver, stomach, and blood in the nude mouse after 4 weeks total exposure to ³H-thymidine. The relative amounts of ³H-thymidine, thymidine, and water in blood, liver, stomach, and tumor tissue of the mice at the end of the treatment regimen are also measured.

A total of thirty 4-6 week old Nu/Nu female nude mice (Charles River Laboratory, Wilmington, Mass.) are used: 10 mice as untreated controls and 0.20 mice subjected to the ³H-thymidine treatment. In the treatment and control groups, 10×10⁶ BT-20 human breast cancer cells are inoculated subcutaneously into the flanks of the mice. Animal weights are recorded daily and animals are closely monitored for signs of illness. Approximate tumor volumes are measured every other day using calipers to measure the height (H), width (W), and length (L) of each tumor. Tumor volumes are calculated according to the formula for volume of an ellipsoid V=πHWL/6. Tumors grow to 300-500 mm³ in approximately 3-4 weeks after inoculation. A set of 6 measurements per day is made on a single tumor in the control group on one day of each week to determine the standard deviation versus volume in the volume measurements.

When tumors reach 300-500 mm³ in volume, the 20 mice of the treatment group are given intratumoral injections of 2 μCi ³H-thymidine in 20 μl of saline weekly for two weeks. Tumor growth is monitored for a total of 4 weeks, whereupon the mice are sacrificed and the tumors and other tissue are harvested.

Lines are drawn along the anterior-posterior length of the harvested tumors. In addition, the top, left and right of the harvested tumors is marked. The tumors are quartered, and the tip from each quarter that was in the tumor center is removed and combined into one sample. Thus there are 5 tumor samples—one from each quarter and 1 from the tumor center. The issue samples collected from the stomach, liver and blood are also processed. Each tissue sample is completely dissolved in 1-2 ml of tissue solubilizer (Soluene-50, Packard Instruments). A portion of the sample in is placed in Hionic-Fluor LSC cocktail and the radioactivity counted in a scintillation counter.

Thin Layer Chromatography (TLC) of tissue (including tumor and blood) samples from six mice is performed to measure relative amounts of ³H-thymidine, ³H-thymine, and ³H-water in the samples. Samples are prepared for TLC as follows: tissue is cut into 50-100 mg pieces and placed in a glass vial containing 1-2 ml of tissue solubilizer (Soluene-50, Packard Instruments) and incubated at 50-60° C. for approximately 24 hours. After the tissue is solubilized, the solution is cooled to room temperature.

Ten to fifteen microliters of the dissolved tissue or is spotted on a silica gel TLC plate, and the plate is placed in a chromatography chamber containing ˜200 ml of eluant so the dissolved tissue sample is just above the eluant surface. The solvent front is allowed to move for about 2 hours, whereupon the TLC plate is removed from the chamber and stained with iodine. The spots representing ³H-thymidine, ³H-thymine, and ³H-water are scraped from the silica gel and placed in scintillation cocktail. The radioactivity is counted in the scintillation counter.

The concentration of ³H-thymidine, ³H-thymine, and ³H-water in all tissue samples is determined by correcting the count rates for quenching and converting counts to concentration of ³H-thymidine from a standard. For the tumor samples, ³H-thymidine concentration is considered separately for each of the 5 pieces and also as two different groups; mean of the center sample versus the mean of the outer (4 quarters), and mean of the of all 5 samples for each tumor. The data is blocked by replicate to minimize inherent variability in the measurements deriving from the scintillation counting technique and the heterogeneity of the tumors.

The same scintillation procedure is followed for determining amounts of ³H-thymidine, ³H-thymine, and ³H-water measured by TLC. These amounts are used to determine relative concentrations of these compounds for each of the sampled tissue types. These data are also blocked by replicate.

Tumor growth is also analyzed by calculating the percent reduction in tumor volume over the course of the experiment. The error in this measurement is determined from the measured standard deviation and the standard formula for calculating the error for a percent difference. Systemic toxicity is not directly measured, but any signs of illness are recorded. Weight is also monitored and plotted daily.

Slight variations in the above described experimental design are not expected to significantly change the anticipated results.

Anticipated Results—At 4 weeks post injection, it is expected that about 10% of the injected ³H-thymidine dose will be retained in the tumor and distributed evenly throughout the tumor tissue. It is likely that no measurable ³H-thymidine will be found in other tissues. Tumors will be less than about 20% of their initial volume at 4 weeks post-injection.

Intratumoral injection is expected to significantly increase the achievable concentration of ³H-thymidine in tumors and minimize the systemic ³H-thymidine concentration. This is because most of the ³H-thymidine will likely be retained in the tumor and the slow “leaching” of ³H-thymidine into the blood would allow the agent to be metabolized into ³H-thymine which is not incorporated into DNA, and thus has little or no cytotoxicity.

### EXAMPLE 5

### Dose Response Characteristics for Intratumoral and Intravenous Injections of ³H-Thymidine

BT-20 human breast cancer tumors are grown in the flanks of six experimental groups of twenty nude mice as in Example 3. The tumors are grown to volume of approximately 500 mm³. One group of ten mice serves as untreated controls. The average weight of the experimental and control mice is 20 grams.

In each of the six experimental groups, ten mice receive a given ³H-thymidine dose by direct injection into a tumor, and ten mice receive the same dose by tail-vein injection. Each experimental group is dosed only once. The doses are represented in Table 3 as total amount of ³H-thymidine given (μCi/20 μl saline), the equivalent dose based on approximate tumor mass (μCi/gram of tumor) and the equivalent dose based on approximate body weight (μCi/kg body wt.). Equivalent doses for a 60 kg human calculated from Table 2 above are given in parentheses in the “dose μCi/kg body wt.” column.

TABLE 3
Doses for administration to
nude mice carrying human xenograft tumors.

| | amount ³H-thymidine | dose | dose |
|---|---|---|---|
| Group | (μCi/20 μl saline) | μCi/gram of tumor | μCi/kg body wt. |
| 1 | 2 | 4 | 100(8.33) |
| 2 | 4 | 8 | 200(16.67) |
| 3 | 8 | 16 | 400(33.33) |
| 4 | 16 | 32 | 800(66.67) |
| 5 | 32 | 64 | 1600(133.33) |
| 6 | 50 | 100 | 2500(208.33) |

The mice are monitored daily, and the tumors measured every two days for a minimum of two weeks or until tumor regrowth begins. The data are expressed as ratios of the tumor volume at any given time point to its pre-treatment volume. Tumor measurements are normalized for variations in initial volume by calculating the results as:

r=Sₜ/Sᵢ

where r is the ratio of tumor volume at a given time point (Sₜ) to initial tumor volume (Sᵢ). Note that “r” is the inverse of ratio “R” used in Example 2 above (R is equivalent to Sᵢ/Sₜ). Thus, if no growth occurred, the ratio r would be 1. For tumor remission r would be less than 1, and for tumor growth r would be greater than 1. An r of 0 indicates total regression of the tumor.

Slight variations in the above described experimental design are not expected to significantly change the anticipated results.

Anticipated Results for Systemic Injection: It is expected that little to no tumor growth will be seen after systemic doses of 100, 200 or 400 μCi/kg body weight. Moderate to medium tumor regression will be seen in systemic doses of 800, 1600 and 2500 μCi/kg body weight (see Table 4 below). As above, equivalent human doses are given in parentheses.

TABLE 4
Anticipated approximate regression of human xenograft tumors in nude
mice injected systemically with the indicated dose of ³H-thymidine.

| dose | |
|---|---|
| (μCi/kg body wt.) | r |
| 100(8.33) | 1.0 |
| 200(16.67) | 1.0 |
| 400(33.33) | 1.0 |
| 800(66.67) | 0.9 |
| 1600(133.33) | 0.8 |
| 2500(208.33) | 0.6 |

Anticipated Results for Intratumoral Injection: It is expected that tumor regression will be observed at all doses, with significant reduction at doses of 16 μCi/gram tumor and above. Total regression of the injected tumor is likely at doses of 32, 64 and 100 μCi/gram tumor (see Table 5 below).

TABLE 5
Anticipated approximate regression of human xenograft tumors in nude
mice injected intratumorally with the indicated dose of ³H-thymidine.

| dose | |
|---|---|
| (μCi/gram tumor) | r |
| 4 | 0.8 |
| 8 | 0.6 |
| 16 | 0.2 |
| 32 | 0 |
| 64 | 0 |
| 100 | 0 |

### EXAMPLE 6

### Pharmacokinetics of Intratumoral Administration of ³H-Thymidine

Unlike external beam irradiation and conventional brachytherapy, where the irradiation dose is easily controlled, the dose from intratumorally injected ³H-thymidine to the tumor and other bodily systems is dependent on biological processes. Thus, to determine the most efficacious protocol for a multiple intratumoral injection of ³H-thymidine, a pharmacokinetic model describing the transport of ³H-thymidine in the single dose case is utilized. Using the theoretical half-life of ³H-thymidine in the tumor from Example 7 below, and the theoretical dose-response data from Example 8 below, the superposition method of Sharget and Yu, Applied Biopharmaceutics and Pharmacokinetics, Appleton-Century-Crofts, New York, N.Y., 1980, is used to extend the two compartment model from the single dose to the multidose case.

The two-compartment first-order linear pharmacokinetic model chosen describe the ³H-thymidine transport from a single intratumoral dose is taken from Talarida, Manual of Pharmacologic Calculations with Computer Programs, Springer-Verlag, New York, N.Y., 1987, and is represented schematically below:

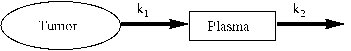

The two compartments are the tumor and the blood plasma, respectively. The model includes three key parameters Tₒ, k₁, and k₂, where

Tₒ is the initial amount of ³H-thymidine found in the tumor nuclei; and

k₁ and k₂ are diffusion constants which describe diffusion of ³H-thymidine out of the tumor into the plasma, and then out of the plasma, respectively.

The model can be described using Equations 1 and 2:

dT/dt=−k₁T Equation 1

dP/dt=k₁T−k₂P Equation 2

In Equations 1 and 2:

k₁ and k₂ are as defined above;

T is time in a particular compartment (tumor or plasma);

P is the amount of ³H-thymidine in the plasma;

T is the amount of ³H-thymidine in the tumor;

dT/dt is the change in the amount of ³H-thymidine in the tumor over time;

dP/dt is the change in the amount of ³H-thymidine in the plasma over time.

The model gives an exponential decay in time for the clearance of a single dose from the tumor, with a half-life (t_{1/2}) given by:

t_{1/2}=ln(2)/k₁ Equation 3

### EXAMPLE 7

### Half-Life of ³H-thymidine in Human Xenograft Tumors Carried by Nude Mice

To measure the time course of the retention of the ³H-thymidine in the tumor xenografts, 180 4-6 week old Nu/Nu female nude mice (Charles River Laboratory, Wilmington, Mass.) are divided into three groups of 60 mice each, and each group is treated at a different dose. BT-20 tumor cells (10 million cells in 0.2 ml PBS) are inoculated into the right flank of each mouse as in Example 4 above. The tumors are grown to volumes of 300-500 mm³ over the course of 3-4 weeks.

After tumors in all mice have reached the required 300-500 mm³ volume, each group of 60 is divided into ten subgroups of six animals each. As the tumors have variable growth rates, the subgroups will be chosen such that the mean tumor volume and standard deviations are similar. ³H-thymidine, specific activity 89.9 Ci/mmol, (New England Nuclear Life Sciences Products, Boston, Mass.), in 20 μl of saline is injected into the tumors on each mouse at a dose of 4 μCi/g of tumor (group I), 16 pCi/g of tumor (group II), and 50 μCi/g tumor (group III).

One subgroup of six mice from each group is sacrificed by asphyxiation at the following time points: 24 hours post-injection, and then every three days for the next 28 days (a total of 10 time points). The time points have been chosen according to the known bi-exponential decay governing systemic ³H-thymidine excretion (Straus M J et al (1977), supra). As the long time constant from the Straus model is more relevant here, the first point (i.e. 24 hours) is greater than five times the short (less than 1 hour) half-life reported by Straus. The last data point (˜28 days) is roughly 3 times the long (10.8 day) half-life reported by Straus. The other time points are taken at an equal distribution in between.

The standard deviation in the ³H-thymidine measurements is likely similar to the EMT sarcoma ³H-thymidine uptake data in BALB/C mice (see Larson S M et al., [1980] Radiology 134(3): 771-773), and can be conservatively estimated at 10%. Thus, 6 mice per time point at ten time points is adequate to determine the half-life.

Levels of ³H-thymidine are determined by liquid scintillation counting for each mouse in tumor, blood, liver, stomach, and small intestine samples. To measure the distribution and retention of ³H-thymidine in the tumors, each tumor is divided into 8 sections by bisecting the lesion three times. The tip of each section corresponding to the tumor center is sliced off and these pieces are pooled. The level of ³H-thymidine is determined separately for each of the 9, (i.e. 8+center) sections for each tumor as in Example 3 above.

The half-life for retention of ³H-thymidine in the tumors is measured by fitting the concentration of ³H-thymidine in the tumor samples at each time point to a theoretical exponential curve predicted by the pharmacokinetic model outlined above. Half-life is determined separately for three different divisions of tumor tissue: whole tumors, tumor outer pieces, and tumor centers, to check for effects that may be caused by the vascular distribution. The error for each point is the standard deviation in the measurement. The half-life for the three groups of six animals per subgroup and ten time periods is analyzed using two-way ANOVA.

Reduction in tumor volume is estimated as in Example 4 for each treatment subgroup over the course of the experiment. The data is analyzed by calculating the percentage of tumor reduction as in Example 4. The error in this measurement is determined from the measured standard deviation and the standard formula for calculating the error for a ratio given above in Equation 1. Systemic toxicity is not directly measured, but is monitored as described above in Example 4.

Slight variations in the above described experimental design are not expected to significantly change the anticipated results.

Anticipated Results—It is expected that the half-life for ³H-thymidine retention in the human xenograft tumors will be approximately 4 days for all three groups. The half-life of ³H-thymidine in the tumors is likely not dose dependent over the range of doses from 4 μCi/g tumor to 50 μCi/g tumor, but is a function of tumor volume.

It is expected that there will be a significant reduction in tumor volume for each treatment subgroup.

### EXAMPLE 8

### Determination of a Preliminary in vivo Dose-Response Curve and Determination of the Tumor Toxicity of Single Intratumoral Injections of Escalating Amounts of ³H-Thymidine

Six experimental groups of 18 4-6 week old Nu/Nu female nude mice (Charles River Laboratory, Wilmington, Mass.) are used. In each of the six groups an additional group of 6 mice serves as an uninoculated control. Thus the total number of experimental mice is 108 and total number of uninoculated control mice is 36.

Human BT-20 breast cancer tumors are grown in the flanks of all experimental mice as in Example 4 above. Tumors are grown to an approximate volume of 300-500 mm³ over the course of 3-4 weeks. Each experimental group is then divided into two subgroups: one treatment subgroup of 12 mice and one untreated (but inoculated) control subgroup of six mice. This experimental setup provides 80% power assuming the same standard deviation in the groups as seen in the previous examples. Because the tumors have variable growth rates, subgroups are chosen such that the mean tumor volume and standard deviation are similar for each subgroup.

³H-thymidine in normal saline is injected into the tumors of each treatment subgroup of each group as follows:

| | amount ³H-thymidine | dose |
|---|---|---|
| Group | (μCi/20 μl saline) | (μCi/gram of tumor) |
| 1 | 1 | 2 |
| 2 | 2 | 4 |
| 3 | 4 | 8 |
| 4 | 8 | 16 |
| 5 | 16 | 32 |
| 6 | 25 | 50 |

The mice are monitored daily and the tumors measured daily by microcaliper for a minimum of 3 weeks or until tumor re-growth begins. Tumor volumes are calculated as in Example 2. Three daily tumor volumes are measured for the first group so that the error in the volume measurement can be determined as in Example 4. The mean volumes and standard deviations are calculated and growth rate curves are monitored during the experiment. The data are expressed as percent difference of the tumor volume at any given time point to its pre-injection volume.

Tumor volume data is analyzed as described above in the in Example 4 by calculating percent tumor reduction and initial tumor volume. Systemic toxicity is not directly measured, but is monitored as described above in Example 4. Concentrations and distribution of ³H-thymidine in the tumors are determined at the end of treatment as described in Example 4.

Slight variations in the above described experimental design are not expected to significantly change the anticipated results.

The dose response curve, which plots the mean percent tumor reduction versus dose, is calculated from the tumor volume measurements. From this curve the ED₉₀, which is the minimal effective dose for eradicating tumors in 90% of the treated mice, is found. The above example gives a theoretical ED₉₀ of 16 μCi/g tumor, which is used as the dose for the multiple administration regimen of Example 9 below.

### EXAMPLE 9

### Development and Efficacy of a Multiple Dose Regimen

An intratumoral multiple dose regimen is determined using the theoretical half-life of ³H-thymidine retention in tumors from Example 7 and the theoretical ED₉₀ from Example 8. To accomplish this, the superposition method of Sharget and Yu, supra, is used to extend the two compartment pharmacokinetic model presented in Example 6 from the single dose to the multidose case. This assumes that previous doses do not affect the pharmacokinetics of subsequent doses. Using the superposition method, the maximum and minimum of the multi-dose regimen can be averaged together to give an estimate of the mean concentration in the tumor during multiple injections (Sharget and Yu, supra).

Based on this model, using a theoretical tumor half-life of 4 days and a theoretical ED₉₀ of 16 μCi/g tumor, an effective multiple dose regimen is one intratumoral injection of 16 μCi/g tumor per week, over three weeks. This dose regimen is tested experimentally as follows.

60 4-6 weeks old female nude mice in 2 groups of 30 mice are used. Each group of 30 mice is further divided into two subgroups (10 control mice and 20 treatment mice). As before, the treatment mice will be divided into subgroups that consist of similar distribution of tumor volumes. In each treatment subgroup, 10×10⁶ BT20 human breast cancer cells are inoculated subcutaneously in flanks of the mice as in Example 4. Animal weights are recorded daily and animals are closely monitored for signs of illness. As in Example 4, tumor volumes are measured with a caliper and recorded every other day, and sets of 6 measurements per day are made on a single tumor in the control group on one day of each week to determine the standard deviation versus volume in the volume measurements. Tumors are grown to 300-500 mm³ in volume.

The mice from one treatment subgroup receive an intratumoral injection of 16 μCi/20 μl normal saline (a dose of 32 μCi/gram of tumor). The dose is repeated every 7 days for a total of 3 doses.

To determine the relative efficacy of the same dose injected intravenously, the mice from the other treatment subgroup receive 16 μCi ³H-thymidine by i.v. injection (a dose of ˜800 μCi/kg body wt.) once a week for three weeks.

Slight variations in the above described experimental design are not expected to significantly change the anticipated results.

Anticipated Results—For mice receiving i.v. injection, it is expected that the tumors will show moderate regression (r of approximately 0.8) after three doses. Mice receiving intratumoral injection will show complete and prolonged tumor regression (r of approximately zero).

### EXAMPLE 10

### Preparation of Liposomes Encapsulating Tritiated Nuclear Targeting Agents

Preparation 1

(A) Liposome Preparation

Following the reverse phase evaporation method described in U.S. Pat. No. 4,235,871, liposomes are prepared encapsulating an aqueous solution of 100 μCi/ml ³H-thymidine in normal saline. The liposomes are composed of lactosyl cerebroside, phosphatidylglycerol, phosphatidylcholine and cholesterol in molar ratios of 1:1:4:5. The liposomes so prepared are passed through a 0.4 polycarbonate membrane and suspended in saline, and are separated from non-encapsulated material by column chromatography in 135 mM sodium chloride, 10 mM sodium phosphate pH 7.4. The amount of encapsulated ³H-thymidine is measured by scintillation counting. The liposomes thus prepared are used without further modification, or are modified as described below.

(B) Preparation of Liposome Surface

A quantity of the liposomes prepared in (A) above are charged to an appropriate reaction vessel to which is added a stirring a solution of 20 mM sodium metaperiodate, 135 mM sodium chloride and 10 mM sodium phosphate (pH 7.4). The resulting mixture is allowed to stand in darkness for 90 minutes at a temperature of about 20° C. Excess periodate is removed by dialysis of the reaction mixture against 250 ml of buffered saline (135 mM sodium chloride, 10 mM sodium phosphate, pH 7.4) for 2 hours. The product is a liposome having a surface modified by oxidation of carbohydrate hydroxyl groups to aldehyde groups. Various targeting groups or opsonization inhibiting moieties are conjugated to the liposome surface via these aldehyde groups.

Preparation 2

The procedure of Preparation 1, supra., was repeated, except that in place of ³H-thymidine, there is encapsulated an aqueous mixture of 100 μCi/ml of the c-myc oligonucleotide:

CAC GTT GAG GGG CAT (SEQ ID NO:1)

The cmyc oligonucleotide is synthesized with [methyl,1′,2′-3H]thymidine 5′-triphosphate (Amersham Pharmacia Biotech, Inc., cat. # TRK576).

Preparation 3

Dimyristoylphosphatidylethanolamine (DMPE) (100 mmoles) is dissolved in 5 ml of anhydrous methanol containing 2 equivalents of triethylamine and 50 mg of m-maleimidobenzoyl N-hydroxysuccinimide ester (Kitagawa and Aikawa, J. Biochem. 79,233-236, 1976). The resulting reaction is allowed to proceed under a nitrogen gas atmosphere, at room temperature, overnight. The resulting reaction mixture is subjected to thin layer chromatography on Silica gel H in chloroform/methanol/water (65/25/4), which reveals quantitative conversion of the DMPE to a faster migrating product. Methanol is removed under reduced pressure and the products redissolved in chloroform. The chloroform phase is extracted twice with 1% sodium chloride and the maleimidobenzoyl-phosphatidylethanolamine (MBPE) purified by silicic acid chromatography with chloroform/methanol (4/1) as the solvent. Following purification, thin-layer chromatography indicates a single phosphate containing spot that is ninhydrin negative. The MBPE is an activated phospholipid for coupling sulfhydryl containing compounds, including proteins, to the liposomes.

Preparation 4

The procedures of Preparations 1-2 are repeated using the MBPE from Preparation 3 and phosphatidylcholine and cholesterol in molar ratios of 1:9:8. The vesicles are separated from the unencapsulated tritiated nuclear targeting reagent by column chromatography in 100 mM sodium chloride-2 mM sodium phosphate (pH 6.0).

### EXAMPLE 11

### Attachment of Antibody to the Liposome Surface

An appropriate vessel is charged with 1.1 ml of Preparations 1 or 2, containing 10 mmoles of liposomes. To the charge there is added with stirring 1.0 ml of monoclonal antibody (3.0 mg protein) and 0.2 ml of a 200 mM sodium cyanoborohydride solution. The resulting reaction mixture is allowed to stand overnight while maintained at a temperature of 4° C. At the end of this period of time, the reaction mixture is separated on a Biogel A5M agarose column (Biorad, Richmond, Calif.; 1.5×37 cm).

### EXAMPLE 12

### Attachment of Antibody Following Generation of Aldehyde on the Liposome by Treatment with Galactose Oxidase

To attach the antibody to the surface of liposomes prepared in Preparations 1-2 above, the C-6 hydroxyl group on the galactose residue of the lactosylcerebroside was oxidized to an aldehyde by the enzyme galactose oxidase as detailed by Zile et al., J. Biochem., 254,3547-3553 (1979). 5 mmoles of the liposome from preparation 1 or 2 above is incubated with 25 units of galactose oxidase (Sigma Corp.) in saline for 4 hours at a temperature of 37° C. Liposomes containing an aldehyde group on their surface are separated from the enzyme by column chromatography. The fraction containing the vesicles is mixed with 2 mg of antibody and 0.2 ml of a freshly prepared solution of sodium cyanoborohydride and are reacted at room temperature overnight. The vesicles containing the antibody on their surface are separated from non-attached antibody.

All references discussed herein are incorporated by reference. One skilled in the art would readily appreciate the present invention is well adapted to carry out the stated objects and obtain the disclosed ends and advantages, as well as those inherent herein. The present invention may be embodied in other specific forms without departing from the spirit or essential attributes thereof. Therefore, any descriptions of specific embodiments in the foregoing disclosure is not to be construed as limiting the scope of the present invention.

We claim:

1. A method of treating proliferative disorders, comprising the steps of:
a) providing a subject having tissue associated with a proliferative disorder, wherein said tissue comprises target cells having DNA-containing nuclei;
b) administering an effective amount of a tritiated nuclear targeting agent to said subject so that the target cells are exposed to the tritiated nuclear targeting agent; and
c) allowing said tritiated nuclear targeting agent to be transported to the target cell nuclei such that the tritiated nuclear targeting agent associates with the target cell DNA,
whereupon the tritiated nuclear targeting agent causes target cell death.

2. The method of claim 1 wherein said proliferative disorder is selected from the group comprising cancerous proliferative disorders, hemangiomatosis in the newborn, secondary progressive multiple sclerosis, chronic progressive myelodegenerative disease, neurofibromatosis, ganglioneuromatosis, keloid formation, Pagets Disease of the bone, uterine and breast fibrocystic disease, Peronies and Duputren's fibrosis, cirrhosis, atherosclerosis and vascular restenosis.

3. The method of claim 2 wherein said proliferative disorder comprises a cancerous proliferative disorder.

4. The method of claim 2 wherein said proliferative disorder comprises vascular restenosis.

5. The method of claim 3 wherein said tissue comprises a tumor.

6. The method of claim 4 wherein said tissue comprises a restenotic plaque.

7. The method of claim 1 wherein said tritiated nuclear targeting agent is selected from the group consisting of nucleic acid precursors, steroid hormones and oligonucleobases.

8. The method of claim 7 wherein said tritiated nuclear targeting agent is a nucleic acid precursor.

9. The method of claim 8 wherein said nucleic acid precursor is selected from the group consisting of adenosine, cytidine, guanosine, thymidine, and uridine.

10. The method of claim 9 wherein said nucleic acid precursor is thymidine.

11. The method of claim 5 wherein said tritiated nuclear targeting agent is a nucleic acid precursor.

12. The method of claim 11 wherein said nucleic acid precursor is thymidine.

13. The method of claim 7 wherein said tritiated nuclear targeting agent is a steroid hormone.

14. The method of claim 13 wherein said steroid hormone is selected from the group consisting of cortisol, estradiol, testosterone, progesterone, tamoxifen and their analogs and derivatives.

15. The method of claim 13 wherein said steroid hormone binds to estrogen receptors.

16. The method of claim 13 wherein said steroid hormone binds to testosterone receptors.

17. The method of claim 7 wherein said tritiated nuclear targeting agent is an oligonucleobase.

18. The method of claim 17 wherein said oligonucleobase is 1-40 nucleobases in length.

19. The method of claim 18 wherein said oligonucleobase comprises the sequence of a proto-oncogene, an oncogene, or IRT-1.

20. The method of claim 19 wherein said oligonucleobase is nuclease resistant.

21. The method of claim 19 wherein said sequence is selected from the group comprising SEQ ID NO:1, SEQ ID NO:2 and SEQ ID NO:3.

22. The method of claim 1 wherein said tritiated nuclear targeting agent is administered by infusion.

23. The method of claim 22 wherein said infusion is intravascular.

24. The method of claim 22 wherein said infusion is subcutaneous.

25. The method of claim 22 wherein said infusion is sustained for a period of time during which the majority of target cells enter the S-phase of the cell cycle.

26. The method of claim 22 wherein said infusion comprises multiple infusions.

27. The method of claim 1 wherein said tritiated nuclear targeting agent is administered by injection.

28. The method of claim 27 wherein said injection comprises injection into the vasculature.

29. The method of claim 27 wherein said injection comprises direct injection into the target tissue.

30. The method of claim 28 or 29 wherein said tritiated nuclear targeting agent is administered by multiple injections.

31. The method of claim 1 wherein said tritiated nuclear targeting agent is administered by direct application.

32. The method of claim 31 wherein said direct application comprises administration by a catheter.

33. The method of claim 6 wherein said tritiated nuclear targeting agent is administered by direct application.

34. The method of claim 33 wherein said direct application comprises administration by a catheter.

35. The method of claim 1 wherein said tritiated nuclear targeting agent is carried by a structure.

36. The method of claim 35 wherein said structure is selected from the group consisting of liposomes, micelles and microcapsules.

37. The method of claim 36 wherein said structure is modified to affect its biodistribution.

38. The method of claim 37 wherein the structure is a modified liposome.

39. The method of claim 38 wherein said modified liposome comprises an opsonization inhibiting moiety.

40. The method of claim 39 wherein said opsonization inhibiting moiety comprises polyethylene glycol.

41. The method of claim 38 wherein said modified liposome comprises a targeting group.

42. The method of claim 41 wherein said targeting group comprises an antibody.

43. The method of claim 42 wherein said antibody is selected from the group consisting of B72.3 antibodies, 9.2.27 anti-melanoma antibodies, D612 antibodies, UJ13A antibodies, NRLU-10 antibodies, 7E11C5 antibodies, CC49 antibodies, TNT antibodies, PR1A3 antibodies, B43 antibodies, and anti-CD105 antibodies, antibodies to vascular smooth muscle cells, and antibodies to the IRT-1 gene product.

44. The method of claim 41 wherein said targeting group comprises the E. coli heat stable enterotoxin ST.

45. A sterile, pyrogen free pharmaceutical composition for treating proliferative disorders comprising a tritiated nuclear targeting agent.

46. The sterile, pyrogen free pharmaceutical composition of claim 45 wherein said tritiated nuclear targeting agent is selected from the group consisting of nucleic acid precursors, steroid hormones and oligonucleobases.

47. The sterile, pyrogen free pharmaceutical composition of claim 46 further comprising a structure for carrying said tritiated nuclear targeting agent.

48. The sterile, pyrogen free pharmaceutical composition of claim 47 wherein said structure is selected from the group consisting of liposomes, micelles and microcapsules.

49. The sterile, pyrogen free pharmaceutical composition of claim 48 wherein said structure is modified to affect its biodistribution.

50. The sterile, pyrogen free pharmaceutical composition of claim 49 wherein said structure is a modified liposome.

51. The sterile, pyrogen free pharmaceutical composition of claim 50 wherein said modified liposome comprises an opsonization inhibiting moiety.

52. The sterile, pyrogen free pharmaceutical composition of claim 51 wherein said opsonization inhibiting moiety comprises polyethylene glycol.

53. The sterile, pyrogen free pharmaceutical composition of claim 50 wherein said modified liposome comprises a targeting group.

54. The sterile, pyrogen free pharmaceutical composition of claim 53 wherein said targeting group comprises an antibody.
